(19) United States
(12) Patent Application Publication
(10) Pub. No.: US 2010/0211048 A1
(43) Pub. Date: Aug. 19, 2010

(54) MEDICAL GUIDE WIRE

(75) Inventors: Tomohisa Arai, Kanagawa-Ken (JP); Fumihiko Yoshimura, Kanagawa-Ken (JP)

Correspondence Address: HARNESS, DICKEY & PIERCE, P.L.C., P.O. BOX 828, BLOOMFIELD HILLS, MI, 48303, US

(73) Assignees: KABUSHIKI KAISHA TOSHIBA, Tokyo (JP); TOSHIBA MATERIALS CO., LTD., Yokohama-Shi, Kanagawa-Ken (JP)

(21) Appl. No.: 12/680,032

(22) PCT Filed: Sep. 19, 2008
(86) PCT No.: PCT/JP2008/066980

§ 371 Date: Mar. 25, 2010

(30) Foreign Application Priority Data
Sep. 25, 2007 (JP) 2007-247579

(51) Int. Cl. A61M 25/09

(52) U.S. Cl. 604/528

(57) ABSTRACT

Disclosed is a medical guide wire that can realize improved insertability and imaging properties. The medical guide wire comprises a medical guide wire main body part comprising a body part and a frontal end part having a smaller wire diameter than the body part, and a coil part and a cap part provided at the frontal end part. The coil part comprises a wire which is a clad wire comprising a core part that is composed mainly of at least one of tungsten and molybdenum and a covering part that covers the core part and is composed mainly of titanium.

### TECHNICAL FIELD

The present invention relates to a medical guide wire for use in the insertion of a catheter mainly into a blood vessel or the like.

### BACKGROUND ART

For example, in angiography and coronary artery treatment, a catheter is inserted into a blood vessel or a treatment site for various types of treatment. Catheters are, for example, in the form of ultrafine tubes or balloons. A medical guide wire is used for safely inserting the catheter into a treatment part such as a blood vessel (the term “guide wire” in the following description referring to “medical guide wire”). The catheter is formed of a highly flexible material and thus cannot be solely inserted, for example, into a blood vessel which is complexly bent without difficulties. Accordingly, a method is adopted in which a medical guide wire is inserted, for example, into a blood vessel and a catheter is inserted along the medical guide wire.

The medical guide wire is inserted into a complexly bent blood vessel and thus is required to be flexible and operable. Further, the medical guide wire used in this application is 10 cm in length and is in some cases as long as not less than 100 cm and thus should have strength high enough to prevent disconnection even when the medical guide wire is in a thin wire form. That is, the medical guide wire is required to have flexibility high enough to be insertable into a blood vessel having a complicated shape and, at the same time, have strength high enough to prevent disconnection even when the medical guide wire is in a thin wire form. Further, it is of course important that the guide wire does not adversely affect the human body. Furthermore, the guide wire is inserted into a complexly bent blood vessel while being turned and moved backward and forward and thus is required to have torque transmissibility and pushability.

A thin wire formed of stainless steel or a thin wire formed of an Ni—Ti-base superelastic alloy have hitherto been used as the medical guide wire. The thin wire formed of stainless steel has good pushability. The thin wire, however, suffers from a problem, for example, that, when the thin wire is passed into a complexly-shaped blood vessel, for example, strain remains unremoved after the passage of the thin wire into a place having a small radius of curvature, or high resistance occurs when the thin wire is passed into a place having a small radius of curvature. Thus, the flexibility of the thin wire formed of stainless steel is not always satisfactory.

On the other hand, the thin wire formed of Ni—Ti-base superelastic alloy is advantageously flexible and causes no significant resistance when the thin wire is passed into a place having a small radius of curvature. The thin wire formed of Ni—Ti-base superelastic alloy, however, disadvantageously has a very low Young's modulus and a large hysteresis in a stress-strain curve, and, thus, the torque transmissibility is not always good.

In order to solve these problems, Japanese Patent Application Laid-Open No. 111849/2003 (hereinafter referred to as “patent document 1”) provides a composite thin wire comprising a superelastic titanium alloy wire formed of an Ni—Ti alloy and a stainless steel wire that have been interwoven into each other. Further, Japanese Patent Application Laid-Open No. 337361/2004 (hereinafter referred to as “patent document 2”) proposes a guide wire comprising a core wire formed of a superelastic alloy and a plastic metal covering the circumference of the core wire. Specifically, in this guide wire, an Ni—Ti alloy is used as a core wire, and the core wire is covered, for example, with copper plating.

Both the techniques disclosed in patent documents 1 and 2 are of a type that uses a superelastic alloy as a core material. The superelastic alloy material, even when significantly deformed, restores its original shape upon the stop of application of force and causes no significant stress upon deformation. Accordingly, highly flexible guide wires can be manufactured from the superelastic alloy.

The interweaving type disclosed in patent document 1, however, is disadvantageous in that there is a limitation on a reduction in diameter of the guide wire. The adoption of the superelastic alloy as the core material can contribute to improved flexibility, but on the other hand, is not always satisfactory in operability. Further, the superelastic alloy generally has a hysteretic stress-strain curve and cannot be said to have satisfactory torque transmissibility. That is, the superelastic alloy is disadvantageous in that, in turning operation, play occurs in turning angle upon reverse turning. The titanium-base alloy is permeable to X-rays, and, thus, the blood vessel at its position into which the guide wire has been inserted cannot be confirmed by an X-ray transmission image.

Passing the guide wire into a complexly shaped blood vessel means that the guide wire is inserted while repeating both bending and shape restoration. In the conventional guide wire, when the length is less than 20 cm, no problem occurs. However, as the length increases, the operability is deteriorated and becomes unsatisfactory, disadvantageously leading to a problem that the guide wire cannot be smoothly inserted while repeating both bending and shape restoration.

Further, it is important that the medical guide wire has good insertability because the medical guide wire is inserted, for example, into a thin blood vessel. In order to improve the insertability, patent document 2 proposes the provision of a coil part (a spring coil) and a cap part (a ball part) at the frontal end part of the medical guide wire. The provision of the coil part and the cap part can contribute to improved insertability but does not always provide satisfactory properties.

When the medical guide wire is formed of the Ni—Ti alloy, the guide wire cannot be observed by an X-ray transmission image without difficulties. Therefore, even when the medical guide wire is inserted into the human body or the like, the position into which the medical guide wire has been inserted cannot be specified by X-ray transmission image observation without difficulties.

Patent document 1: Japanese Patent Application Laid-Open No. 111849/2003

Patent document 2: Japanese Patent Application Laid-Open No. 337361/2004

### DISCLOSURE OF INVENTION

### Problem to be Solved by the Invention

Thus, the conventional medical guide wires have unsatisfactory insertability. Further, in the conventional medical guide wires, difficulties are experienced in specifying the position of the frontal end part of the medical guide wire by an X-ray transmission image. Further, the medical guide wires give priority to an improvement in flexibility and is unsatisfactory in torque transmissibility. Furthermore, both the pushability and the torque transmissibility are deteriorated and become unsatisfactory with an increase in the length of the guide wire.

The present invention has been made with a view to solving these problems, and an object of the present invention is to provide a medical guide wire having improved insertability by providing a coil part formed of a predetermined clad wire at the frontal end of a medical guide wire.

Another object of the present invention is to provide a medical guide wire that can realize high torque transmissibility and high pushability by using a predetermined clad wire in a body part in a guide wire.

### Means for Solving Problem

According to one aspect of the present invention, there is provided a medical guide wire comprising: a medical guide wire main body part comprising a body part and a frontal end part having a smaller wire diameter than the body part; and a coil part and a cap part provided at the frontal end part, characterized in that the coil part comprises a wire which is a clad wire comprising a core part that is composed mainly of at least one of tungsten and molybdenum and a covering part that covers the core part and is composed mainly of titanium.

Preferably, a solid solution comprising at least one of tungsten and molybdenum and titanium is present at the boundary between the core part and the covering part.

Preferably, a solid-solution layer comprising at least one of tungsten and molybdenum and titanium is present at the boundary between the core part and the covering part.

The thickness of the solid-solution layer is preferably not less than 0.003 time of the wire diameter in the coil part.

Preferably, the coil part comprises the clad wire wound by three or more turns.

Preferably, the core part is formed of a tungsten alloy containing at least one of rhenium, iridium, rhodium, and ruthenium.

Preferably, the covering part is formed of a titanium alloy comprising at least one of superelastic titanium alloys, α-titanium alloys, αβ titanium alloys, or β-titanium alloys.

The young's modulus of the covering part composed mainly of titanium is preferably not more than 140 GPa. The young's modulus of the core part composed mainly of tungsten or molybdenum is preferably not less than 327 Gpa.

The wire diameter of the coil part, D1, is preferably not more than 0.05 mm. The wire diameter ratio between the coil part and the core part, D2/D1, is preferably in the range of 0.1 to 0.9 wherein D1 represents the wire diameter of the coil part; and D2 represents the wire diameter of the core part.

Preferably, the wire diameter of the body part is not more than 0.5 mm, and the length of the body part is not less than 30 cm.

Preferably, the body part constituting the medical guide wire main body part is a clad wire comprising a core part that is composed mainly of at least one of tungsten and molybdenum and a covering part composed mainly of titanium.

### EFFECT OF THE INVENTION

According to the present invention, the provision of a coil part formed of a predetermined clad wire at the frontal end part can improve the insertability of the medical guide wire. Further, the use of tungsten or molybdenum in a clad wire constituting a coil part can facilitate specifying the position of the medical guide wire by an X-ray transmission image.

Furthermore, the use of a predetermined clad wire in a body part can provide a medical guide wire having high torque transmissibility and pushability. By virtue of these advantages, even thin and/or long guide wires can have excellent properties.

### BRIEF DESCRIPTION OF THE DRAWINGS

FIG. 1 is a diagram showing one embodiment of a medical guide wire according to the present invention.

FIG. 2 is a cross-sectional view showing one embodiment of a coil part according to the present invention.

FIG. 3 is a cross-sectional view showing another one embodiment of a coil part according to the present invention.

FIG. 4 is a diagram showing one embodiment of a body part in a medical guide wire according to the present invention.

FIG. 5 is a diagram showing one embodiment of a medical guide wire main body part according to the present invention.

FIG. 6 is a diagram showing one embodiment of a test evaluation device in a working example of the present invention.

FIG. 7 is a diagram showing one embodiment of the results of evaluation of torque transmissibility of Example 8.

FIG. 8 is a diagram showing one embodiment of the results of evaluation of torque transmissibility of Example 24.

### DESCRIPTION OF REFERENCE CHARACTERS

1 . . . medical guide wire
2 . . . body part
3 . . . frontal end part
4 . . . coil part
5 . . . cap part
6 . . . covering part
7 . . . core part
8 . . . solid-solution layer
9 . . . resin tube
10 . . . input part
11 . . . output part

### BEST MODE FOR CARRYING OUT THE INVENTION

The medical guide wire according to the present invention comprises: a medical guide wire main body part comprising a body part and a frontal end part having a smaller wire diameter than the body part; and a coil part and a cap part provided at the frontal end part, characterized in that the coil part comprises a wire which is a clad wire comprising a core part that is composed mainly of at least one of tungsten and molybdenum and a covering part that covers the core part and is composed mainly of titanium.

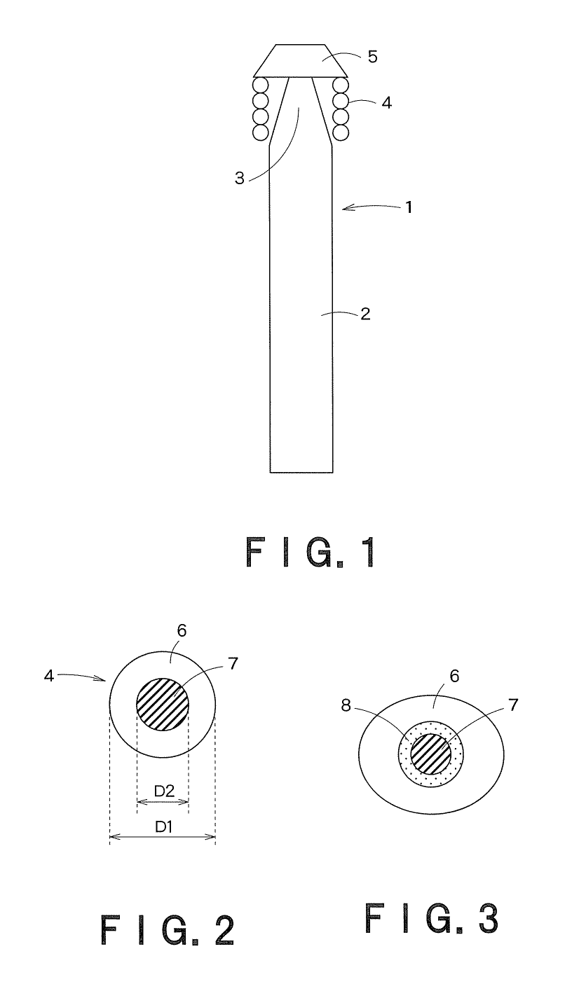

FIG. 1 is a cross-sectional view showing one embodiment of a medical guide wire according to the present invention. In FIG. 1, numeral 1 designates a medical guide wire, numeral 2 body part, numeral 3 frontal end part, numeral 4 a coil part, and numeral 5 a cap part.

The medical guide wire according to the present invention comprises a guide wire main body part comprising a body part and a frontal end part having a smaller wire diameter than the body part. A coil part 4 and a cap part 5 are provided at the frontal end part. In FIG. 1, the cross section of the cap part is trapezoidal. The cross section of the cap part may also be, for example, semicircular or conical. Preferably, the cap part is formed of, for example, an organic material such as resin or rubber or a metallic member.

FIGS. 2 and 3 are cross-sectional views each showing one embodiments of the clad wire constituting the coil part. In the drawings, numeral 6 designates a covering part, numeral 7 a core part, and numeral 8 a solid-solution layer. D1 designates a diameter (outer diameter) of the clad wire constituting the coil part, and D2 a wire diameter (outer diameter) of the core part.

The clad wire constituting the coil part comprises a core part 7 and a covering part 6 and a structure comprising the covering part covering the circumference of the core part. The core part is composed mainly of at least one of tungsten (W) or molybdenum (Mo), and the covering part is composed mainly of titanium (Ti). That is, in the clad wire, the core part is formed of a material having a high Young's modulus, and the covering part is formed of a material having a low Young's modulus. A wire of which the mechanical anisotropy between the radial direction and the axial direction has been set to a desired value can be manufactured by properly selecting the difference in Young's modulus between the core part and the covering part and the component proportion ratio.

A material composed mainly of at least one of tungsten and molybdenum may be mentioned as the material constituting the core part. Examples thereof include tungsten as a simple substance, doped tungsten or tungsten alloy, molybdenum as a simple substance, and doped molybdenum or molybdenum alloy. The expression “composed mainly of tungsten” as used herein means that the content of tungsten in the material is the highest in terms of weight ratio. This is true of “composed mainly of molybdenum.” The tungsten alloy is preferably a rhenium-containing tungsten alloy (Re—W alloy). The Re—W alloy is preferably Re—W alloy having a Re content of 0.2 to 30% by weight. The Re—W alloy has better ductility than tungsten as a simple substance and thus can improve the strength. The Re content is more preferably 2 to 27% by weight from the viewpoint of improved ductility. Other tungsten alloys include those containing 0.2 to 30% by weight of at least one of iridium (Ir), rhodium (Rh), and ruthenium (Ru). The incorporation of at least one of iridium, rhodium, and ruthenium can improve the modulus of elasticity. When the content of at least one of iridium, rhodium, and ruthenium is more than 30% by weight, the workability is deteriorated.

The doped tungsten is tungsten containing a doping agent such as Al (aluminum), Si (silicon), or K (potassium), possesses improved durability at elevated temperatures, and thus can easily be worked into a thin wire, for example, by wire drawing which will be described later. The material composed mainly of tungsten may have an unavoidable impurity content of not more than 1% by weight.

The molybdenum alloy may contain, for example, 0.05 to 1% by weight of at least one of a transition metal such as tin (Sn) and cobalt (Co). The doped molybdenum is molybdenum containing a doping agent such as K (potassium), possesses high durability at elevated temperatures, and recrystallization heat treatment can improve the ductility of the doped molybdenum. The material composed mainly of molybdenum may have an unavoidable impurity of less than 0.05% by weight.

Further, an alloy containing both tungsten and molybdenum is also applicable. When the material contains both tungsten and molybdenum, the total content of tungsten and molybdenum is preferably not less than 50% by weight.

A material composed mainly of titanium may be mentioned as the material constituting the covering part. Examples thereof include titanium as a simple substance and titanium alloys. The expression “composed mainly of titanium” as used herein means that the content of titanium in the material is the highest in terms of weight ratio. The titanium alloy is a superelastic titanium alloy or at least one alloy selected from α-titanium alloy, β-titanium alloy, and α+β-titanium alloy. A nickel-containing titanium alloy (Ni—Ti alloy) may be mentioned as an example of the superelastic titanium alloy. Titanium alloys such as Al(6 atomic %)−V(4 atomic %)−Ti(balance) may be mentioned as an example of the α-titanium alloy, β-titanium alloy, and α+β-titanium alloy. The material composed mainly of titanium may have an unavoidable impurity content of not more than 1% by weight.

Preferred titanium alloys include Ni—Ti alloys or n-titanium alloys. All of these alloys have high workability and can realize easy cladding of the core part therewith. Ni—Ti alloys include a binary alloy comprising Ti as a main component with the balance consisting of Ni (less than 10 to 50% by weight) and a ternary alloy comprising Ti as a main component with the balance consisting of Ni and further 1 to 20% by weight of Mg (manganese), Co (cobalt), Cu (copper) or the like. The β-titanium alloy is an alloy composed mainly of β phase.

The Ni—Ti alloys and a part of β-titanium alloys are superelastic titanium alloys. The “superelasticity” is such a phenomenon that, even when a material is deformed by stress in a particular temperature range, the deformed shape of the material is returned to the original shape when the stress is removed (see “Iwanami Rikagaku Jiten (Iwanami Dictionary of Physics and Chemistry), 5th Edition”). The superelastic alloy generally has a low elastic modulus (Young's modulus) of not more than 100 GPa and thus is a preferred material as the material for the covering part. The stress-strain curve, however, is significantly hystereric, and, thus, the superelastic alloy adversely affects the torque transmissibility. Accordingly, an excessively large relative thickness of the covering part is unfavorable.

As will be described later, when a solid solution of titanium and tungsten or molybdenum is formed, a solid solution of titanium and tungsten or molybdenum may be used as the titanium alloy. The solid solution, even when titanium does not constitute the main component of the solid solution, is regarded as a kind of titanium alloy and as a part of the covering part.

Thus, when the clad wire comprises a core part composed mainly of tungsten or molybdenum and a covering part composed mainly of titanium, the medical guide wire has improved insertability. Since tungsten or molybdenum is used in the core part, the clad wire can be clearly confirmed by an X-ray transmission image. Accordingly, when the medical guide wire is inserted, for example, into the human body, the position of the frontal end part in the medical guide wire can easily be confirmed by an X-ray transmission image.

The young's modulus of pure tungsten, the young's modulus of pure molybdenum, and the young's modulus of pure titanium are 403 GPa, 327 GPa, and 114 GPa, respectively. When the core part is formed of tungsten, molybdenum, or an alloy of tungsten or molybdenum having a high modulus of elasticity while the covering part is formed of titanium having a low modulus of elasticity, a spring effect can be attained, contributing to improved insertability. The young's modulus of the core part and the young's modulus of the covering part are preferably not less than 300 GPa and not more than 140 GPa, respectively. A useful effect can be attained when the difference in Young's modulus between the core part and the covering part is not less than 120 GPa, more preferably not less than 200 GPa.

Preferably, a solid solution comprising a combination of titanium with tungsten or molybdenum, or both tungsten and molybdenum is present at the boundary between the core part and the covering part. Preferably, the solid solution is in a β phase form. The conversion to the β phase can improve the elastic deformation capability and can improve the reliability of the joint area. The solid solution is preferably present as a solid-solution layer.

The whole of a combination of tungsten or molybdenum with titanium can be brought to a solid solution form. Regarding the phase diagram of W and Ti, and Mo and Ti, see “The Moffatt Collection Handbook of Binary Phase Diagrams (published by Genium Publishing Corporation).”

In the manufacture of the clad wire, a solid solution can be formed by holding at a given temperature. The formation of the solid solution results in further increased ductility and thus can improve the strength and workability. When the solid solution is formed in a layer form and has substantially a three-layer structure of core part/solid-solution layer/covering part, an inclined composition is provided and, thus, the ductility can be further improved.

The thickness of the solid-solution layer is not particularly limited. Preferably, however, the thickness is 0.1 to 100 μm or is not less than 0.003 time the outer diameter of the clad wire. Before working to a final wire diameter, the core part and the covering part are joined to each other. In the course of the process, if at least the thickness of the solid-solution layer is less than 1 μm when the outer diameter of the wire is 0.5 mm, disadvantageously, the joint strength of the interface is small. The thickness of the solid-solution layer may exceed 100 μm. In this case, however, the level of irregularities of the surface of the core part is so large that the strength and reliability are lowered and, at the same time, the process control for solid-solution layer formation becomes complicated. Accordingly, the thickness of the solid-solution layer is preferably not more than 100 μm. The thickness of the solid-solution layer decreases with decreasing the wire diameter D1 from 0.05 mm. Therefore, when a wire diameter D1 of not more than 0.05 mm is contemplated, the thickness of the solid-solution layer is preferably not less than 0.003 time the wire diameter D1.

A core wire for a guide wire having the function of the present invention can also be manufactured by, in the manufacture of the clad wire, using pure titanium or a low-Young's modulus titanium alloy as the covering part and, after cladding, heat treating the assembly to diffuse tungsten or molybdenum in the core part in the pure titanium or low-Young's modulus titanium alloy layer for alloying and thus to transform a part or the whole of the covering part into a β phase.

In the solid solution, there are various compositions such as α-Ti and β-Ti. The solid solution, however, is preferably in the form of a single phase of β-Ti. The single phase of β-Ti can provide a solid solution that is chemically stable and highly ductile. The presence or absence of the solid solution can be determined by the surface analysis of a cross section of the clad wire by EPMA. When the solid solution has been formed, the outer diameter D2 of the core part is determined by subjecting the cross section of the core wire in the direction of the diameter of the wire to an EPMA surface analysis to specify the area where titanium is absent, and measuring the length of the longest diagonal line as the outer diameter D2 of the core part.

The clad wire having the above construction is coiled and joined to the frontal end part of a medical guide wire to constitute the coil part. The wire diameter D1 of the coil part is preferably not more than 0.05 mm. Preferably, the clad wire is wound at the frontal end part by three turns or more. Imparting a spring function to the coil part can improve the insertability of the guide wire. To this end, preferably, a thin wire is wound by a plurality of turns. The lower limit of the wire diameter D1 is not particularly limited. From the viewpoint of the efficiency of wire drawing, however, the wire diameter D1 is not less than 0.002 mm.

The clad wire comprising the core part and the covering part is also suitable for use in a body part in a medical guide wire. Even when the body part has a small wire diameter of not more than 0.5 mm or even a very small wire diameter of not more than 0.3 mm, high torque transmissibility and pushability can be provided. In other words, a small wire diameter of not more than 0.5 mm or a very small diameter of not more than 0.3 mm is useful for the guide wire.

Likewise, even when the clad wire is applied to a long guide wire having a length L of not less than 30 cm or even a length L of not less than 100 cm, high torque transmissibility and pushability can be realized.

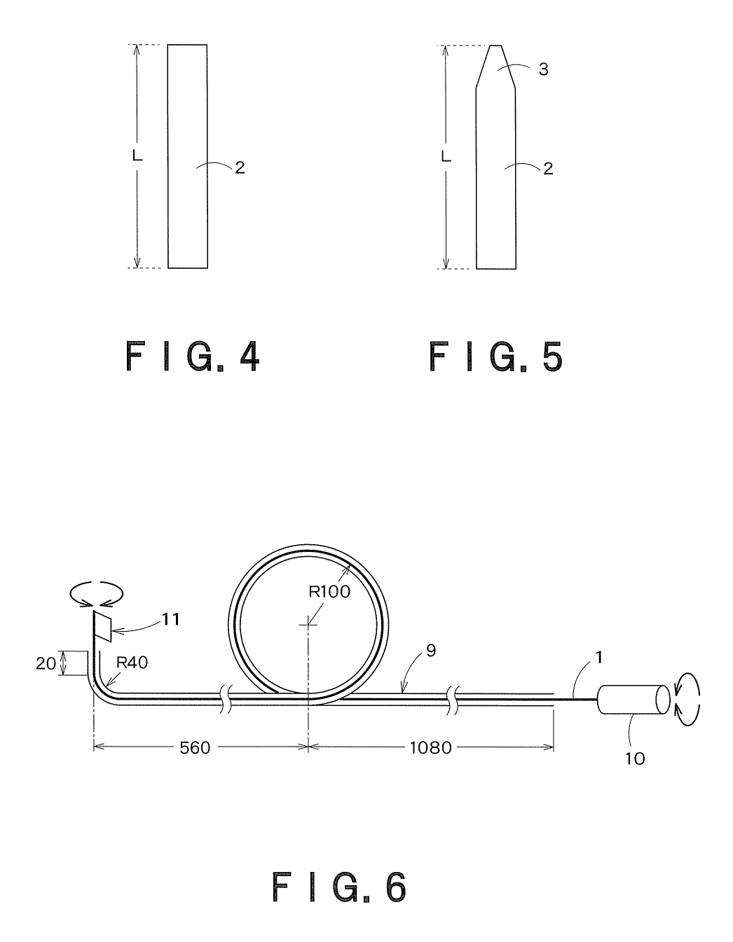

FIG. 4 shows one embodiment of a body part in a medical guide wire according to the present invention. FIG. 4 shows an embodiment of a medical guide wire main body part. In FIG. 4, the medical guide wire main body part has a frontal end part that has been tapered. The medical guide wire main body part having the tapered frontal end part as shown in FIG. 4 is preferred because the guide wire can easily be passed, for example, into a thin hole such as a blood vessel. The frontal end part may be formed of a material different from the material for the core wire (body part). Metal members such as titanium, titanium alloys, and stainless steels and resin members such as hydrophilic resins can be applied as the material for the frontal end part.

The upper limit of the length L of the guide wire is not particularly limited. The length L is preferably not more than 3 m from the viewpoints of insertion into the human body and manufacturability.

The wire diameter ratio between the clad wire and the core part, D2/D1, is preferably in the range of 0.1 to 0.9 wherein D1 represents the wire diameter of the clad wire; and D2 represents the wire diameter of the core part. As described above, the guide wire according to the present invention comprises a core part composed mainly of tungsten, molybdenum, or both tungsten and molybdenum and a covering part composed mainly of titanium. The rigidity which affects the operability and the shape conformability which is flexibility can be improved by regulating the proportion between the tungsten part (or molybdenum part) having a high Young's modulus and the titanium part having a low Young's modulus. In other words, the rigidity and the shape conformability can be regulated by regulating the proportion between the core part composed mainly of tungsten, molybdenum, or both tungsten and molybdenum and the covering part composed mainly of titanium. That is, the rigidity can be further improved by increasing the proportion of the tungsten part having a high modulus of elasticity, and the flexibility can be improved by increasing the proportion of the titanium part. The rigidity can be improved by regulating the proportion (D2/D1) between the wire diameter D1 of the guide wire and the wire diameter D2 of the core part, and, thus, the spring function can be improved.

The D2/D1 value is preferably more than 0.3 and not more than 0.9 from the viewpoint of improving the rigidity and is preferably not less than 0.1 and less than 0.7 from the viewpoint of improving the shape conformability. Further, the D2/D1 value is preferably 0.3 to 0.7 from the viewpoint of providing high strength. In the present invention, the solid solution comprising titanium and tungsten or molybdenum constitutes a part of the covering part.

When the D2/D1 value is less than 0.1 or more than 0.9, the inherent flexibility and operability cannot be satisfactorily ensured and, further, the yield in the manufacture of the wire is lowered.

If necessary, a resin film may be provided on the surface of the guide wire main body part.

The medical guide wire is inserted, for example, into a blood vessel from the coil part-provided side of the medical guide wire. In order to enter a complexly bent blood vessel or the like, the frontal end part should have better insertability. The provision of the coil part can impart a spring function (elasticity) to the frontal end part and thus can improve the operability of the frontal end part.

Next, the method for manufacturing the medical guide wire will be described. The medical guide wire according to the present invention may be manufactured by any method without particular limitation as long as the medical guide wire has the above construction. However, the following manufacturing method is preferred.

At the outset, a rod having a predetermined wire diameter and composed mainly of tungsten, molybdenum, or both tungsten and molybdenum is provided. A titanium tube or a titanium alloy tube into which the rod composed mainly of tungsten, molybdenum, or both tungsten and molybdenum can be inserted is provided.

The rod composed mainly of tungsten, molybdenum, or both tungsten and molybdenum is inserted into the titanium tube or the titanium alloy tube. The titanium tube and the tungsten rod are integrated by hot swaging. In this case, when a rotary swaging machine is used, both the integration and the wire thinning can be performed. Preferably, the outer diameter of the tungsten rod is 1 to 5 mm, and the inner diameter of the titanium tube is approximately (outer diameter of tungsten rod+0.1 mm) to (outer diameter of tungsten rod+2 mm). The wall thickness of the titanium tube is selected according to the final thickness ratio between the core part and the covering part. The formation of a wire having an outer diameter D1 of about 0.8 to 1.5 mm by swaging is preferred. Swaging to an outer diameter of not more than 0.5 mm and even not more than 0.05 mm by the swaging step is also possible. In this case, however, wire thinning only by swaging is likely to cause wire breaking, resulting in lowered yield.

The application of predetermined heat in the swaging step can form a solid solution comprising titanium and tungsten or molybdenum or both tungsten and molybdenum. Further, after the swaging step, a solid solution forming heat treatment step of forming a solid solution by applying heat may be carried out. In order to form a solid solution formed of titanium and tungsten or molybdenum or both tungsten and molybdenum, the adoption of an elevated temperature is advantageous because high-speed ingredient diffusion can be realized and, thus, the treatment can be completed in a short time. However, in this case, embrittlement of the core material is likely to occur. Accordingly, for example, when the solid solution is composed mainly of tungsten, the heat treatment temperature is preferably in the range of 740 to 1200° C. On the other hand, when the solid solution is composed mainly of molybdenum, the heat treatment temperature is preferably in the range of 675 to 1000° C. Further, heating at that temperature for 5 min or longer can result in the formation of a layered solid solution although this depends upon the wire diameter, degree of working and treatment temperature before the heat treatment.

Next, the wire after swaging or solid solution forming heat treatment step can be drawn by a wire drawing step into a thin wire having an outer diameter of not more than 0.5 mm and even not more than 0.05 mm. The wire drawing step for wire thinning can be performed by using a plurality of dies.

A wire as a body part in the guide wire is then provided. The body part wire may be a metal wire formed of titanium, a titanium alloy, stainless steel or the like. The clad wire according to the present invention may also be used as the body part. The torque transmissibility and pushability of the medical guide wire can be advantageously improved by using the clad wire according to the present invention as the body part.

A frontal end part is then provided. The frontal end part may be formed by a method in which the frontal end of the body part is rendered thinner than the body part, for example, by cutting, or alternatively by a method in which a frontal end part which has been previously worked into a rectangular shape is joined, for example, by welding.

A coil part is provided at the frontal end part of the medical guide wire main body part comprising the frontal end part and the body part. The coil part may be formed by joining a wire, which has been previously worked into a coil, for example, by welding or an adhesive, or formed by winding a wire around the main body part for coiling.

Finally, a cap part is provided. The material for the cap part is not particularly limited to a resin or a metal member. However, the cap part formed of a hydrophilic resin is preferred. If necessary, a resin film or a metal plating film may also be provided on the main body part.

The manufacturing method as described above can realize the manufacture of the medical guide wire according to the present invention at a good yield.

### EXAMPLES

A testing evaluation device is shown in FIG. 6. In the drawing, numeral 1 designates a guide wire, numeral 9 a resin tube (made of PTFE) having an inner diameter of 0.6 mm, numeral 10 an input part, and numeral 11 an output part. As shown in the drawing, a testing device 1 comprised a resin tube 9 comprising a linear part having a length of 1080 mm, a circular part having a radius R of 100 mm, a linear part having a length of 560 mm, a curved part having a radius R of 40, and a linear part having a length of 20 mm. A testing device 2 comprised a resin tube 7 comprising a linear part having a length of 200 mm, a circular part having a radius R of 20, a linear part having a length of 200 mm, a curved part having a radius R of 20, and a linear part having a length of 20 mm.

The insertability of the medical guide wire was examined with these testing evaluation devices. The insertability was evaluated as “good,” “somewhat good,” and “failure” in an ascending order of stress applied until the medical guide wire in each of Examples which will be described later is inserted into the resin tube and reaches the output part.

Further, an imaging property was also studied. The imaging property was evaluated as “good” when the image of the frontal end part of the medical guide wire by X-ray transmission after the insertion of the frontal end of the medical guide wire into a beef having a size of 200 mm×200 mm was sharp; was evaluated as “somewhat good” when the image of the frontal end part was somewhat unsharp; and was evaluated as “failure” when the image of the frontal end part was unsharp. The results will be shown below.

### Examples 1 to 7 and Comparative Examples 1 and 2

A clad wire having an outer diameter of 0.32 mm was provided that comprised a core part of doped tungsten (wire diameter: 0.15 mm) and a covering part of pure titanium having a purity of not less than 99.9%.

The frontal end of the clad wire was thinned to form a frontal end part for Examples 1 to 5 and Example 7. Separately, a rectangular frontal end part of pure Ti was provided at the frontal end for Example 6. Next, a coil part (10 turns) was then provided. The coil part was formed of a clad wire (wire diameter: 0.1 mm) comprising a core part of doped tungsten having a wire diameter of 0.0025 to 0.025 mm or doped molybdenum having a wire diameter of 0.025 mm and a covering part of pure titanium. Further, a cap part formed of a hydrophilic resin was provided.

A medical guide wire having the same construction as described above except that the coil part was not provided was provided as Comparative Example 1, and a medical guide wire having the same construction as described above except that the coil part consisted of pure titanium only was provided was provided as Comparative Example 2. These medical guide wires were subjected to the same measurements as described above. The results were as shown below.

TABLE 1

| | Element wire for coil in distal end small diameter part | | | | | | | | | |
|---|---|---|---|---|---|---|---|---|---|---|
| | | | Wire | | | | | | | |
| | | Wire | diameter | | | | | | | |
| | Outer | diameter | in core | | Covering | | Thickness of | Insertability | Insertability | |
| | diameter, | in core part, | part/outer | Core part | part | Specifications of | solid-solution | (testing | (testing | Imaging |
| | mm | mm | diameter | Material | Material | frontal end | layer μm | device 1) | device 2) | property |
| Ex. 1 | 0.05 | 0.025 | 0.5 | Doped W | Pure Ti | Provided with | 0.2 | Good | Good | Good |
| | | | | | | coil and cap | | | | |
| Ex. 2 | 0.05 | 0.025 | 0.5 | 25% Re—W | Pure Ti | Provided with | 0.2 | Good | Good | Good |
| | | | | | | coil and cap | | | | |
| Ex. 3 | 0.05 | 0.015 | 0.3 | Doped W | Pure Ti | Provided with | 0.18 | Good | Good | Good |
| | | | | | | coil and cap | | | | |
| Ex. 4 | 0.05 | 0.005 | 0.1 | Doped W | Pure Ti | Provided with | 0.15 | Good | Good | Somewhat |
| | | | | | | coil and cap | | | | good |
| Ex. 5 | 0.05 | 0.025 | 0.5 | Doped Mo | Pure Ti | Provided with | 0.16 | Good | Good | Somewhat |
| | | | | | | coil and cap | | | | good |
| Ex. 6 | 0.05 | 0.0025 | 0.05 | Doped W | Pure Ti | Provided with | 0.16 | Good | Somewhat | Failure |
| | | | | | | coil and cap | | | good | |
| Ex. 7 | 0.05 | 0.00475 | 0.095 | Doped W | Pure Ti | Provided with | 0.19 | Good | Failure | Good |
| | | | | | | coil and cap | | | | |
| Comp. | — | — | — | — | — | Only tapering | — | Somewhat | Failure | Somewhat |
| Ex. 1 | | | | | | | | good | | good |
| Comp. | 0.05 | — | — | Pure Ti | — | Provided with | — | Good | Somewhat | Failure |
| Ex. 2 | | | | | | coil and cap | | | good | |

As can be seen from the table, the medical guide wires provided with a coil part of the Examples of the present invention had good insertability (testing device 1). For Examples 6 and 7 where the proportion of the core part was small, the spring function of the coil part was so low that the insertability was low in the testing device 2 in which R was small. Further, it was found that, when the core part composed mainly of tungsten or molybdenum was excessively thin, the imaging property was low.

### Examples 8 to 36 and Comparative Examples 3 to 6

Next, the torque transmissibility and pushability of the medical guide wires were measured with the testing device 1. The torque transmissibility and pushability were evaluated as the shape conformability. The torque transmissibility was determined by inserting a guide wire 1 into a resin tube 7 and rotating the guide wire 1 at an input part 9 by 90 degrees and measuring the degree of rotation at an output part 8. Reciprocation rotation was repeated ten times at an angle of +90 degrees to −90 degrees. The same procedure was carried out for 10 samples (n=10). The average of the measured values, the variation (difference between the maximum value and the minimum value), the frequency of discontinuous rotation, and the width of hysteresis were measured.

The travel level of the output part upon the movement of the input part 9 in the front-back direction by 10 mm was measured as the pushability. The pushability was also measured for ten samples ten times per sample, and the average of the measured values was determined. These measurements were performed with an optical angle detecting device and an optical position displacement detecting device.

Guide wires shown in Table 2 were provided as the body part. In the table,

(1) pure W (pure tungsten) refers to tungsten having a W content of not less than 99.9% by weight;

(2) doped W (doped tungsten) refers to pure tungsten containing a doping agent of 30 to 100 ppm;

(3) pure Mo (pure molybdenum) refers to molybdenum having a Mo content of not less than 99.9% by weight;

(4) doped Mo (doped molybdenum) refers to pure molybdenum containing a doping agent of 50 to 100 ppm;

(5) pure Ti (pure titanium) corresponds to class 1 specified in JIS H 4600; and

(6) tungsten alloy, molybdenum alloy, and titanium alloy are alloys respectively having compositions (% by weight) specified in Table 1.

The covering part for Examples 22 to 24 was formed of nitinol (NiTi alloy), the covering part for Examples 25 to 28 was formed of 13% Ta-29% Nb-4.6% Zr—Ti alloy (each percentage being by weight), and the covering part for Example 29 was formed of 6°/0AI-4% V—Ti alloy (each percentage being by weight).

The Ti alloy for Examples 25 to 28 was an alloy that comprised β-Ti as a main phase and had a low Young's modulus of 50 to 80 GPa. The Ti alloy for Example 29 was an alloy that comprised α+βTi— as a main phase and had a Young's modulus of 113 GPa. The Young's modulus of nitinol and the Young's modulus of pure Ti were 100 to 110 GPa and approximately 106 GPa, respectively.

All of pure W, doped W, pure Mo, doped Mo, various W alloys, and Mo alloys had a Young's modulus of not less than 380 GPa.

A rod for constructing a core part (wire diameter: 1 mm) and a tube for constructing a covering part were provided followed by swaging to manufacture a clad wire. Next, the clad wire was optionally heat treated to form a solid-solution layer. Thereafter, wire drawing was performed to a form a guide wire body part having a wire diameter D1 of 0.34 mm. The frontal end of the guide wire body part was then worked into a thin form to form a frontal end part. A coil part using the same clad wire as in Example 1 and a cap part formed of a hydrophilic resin were provided to provide a medical guide wire.

For comparison, a medical guide wire consisting of pure W only was provided as Comparative Example 3, a medical guide wire consisting of pure Ti only was provided as Comparative Example 4, and a medical guide wire consisting of nitinol (NiTi alloy) only was provided as Comparative Example 5. Further, a medical guide wire comprising a core part of nitinol and a covering part of copper plating was provided as Comparative Example 6.

For the medical guide wires of the Examples and the Comparative Examples, the torque transmissibility and pushability were measured by the methods as described above. The results are shown in Table 2.

TABLE 2

| | Guide wire body part | | | | | | |
|---|---|---|---|---|---|---|---|
| | | | Wire | | | | |
| | | Wire | diameter in | | | | Thickness |
| | Outer | diameter | core part/ | | | | of soild- |
| | diameter, | in core | outer | Core part | Covering part | | solution |
| | mm | part, mm | diameter | Material | Material | Properties etc. | layer, μm |
| Ex. 8 | 0.34 | 0.31 | 0.9 | Doped W | Pure Ti | | 1 to 5 |
| Ex. 9 | 0.34 | 0.24 | 0.7 | Doped W | Pure Ti | | 1 to 5 |
| Ex. 10 | 0.34 | 0.17 | 0.5 | Doped W | Pure Ti | | 1 to 5 |
| Ex. 11 | 0.34 | 0.10 | 0.3 | Doped W | Pure Ti | | 1 to 5 |
| Ex. 12 | 0.34 | 0.03 | 0.1 | Pure W | Pure Ti | | 1 to 5 |
| Ex. 13 | 0.34 | 0.17 | 0.5 | Doped W | Pure Ti | | 90 to 100 |
| Ex. 14 | 0.34 | 0.17 | 0.5 | Pure W | Pure Ti | | 0 |
| Ex. 15 | 0.34 | 0.32 | 0.95 | Doped W | Pure Ti | | 1 to 5 |
| Ex. 16 | 0.34 | 0.02 | 0.05 | Doped W | Pure Ti | | 1 to 5 |
| Ex. 17 | 0.34 | 0.17 | 0.5 | Doped W | Pure Ti | | 120 to 200 |
| Ex. 18 | 0.34 | 0.31 | 0.9 | Doped Mo | Pure Ti | | 1 to 5 |
| Ex. 19 | 0.34 | 0.24 | 0.7 | Doped Mo | Pure Ti | | 1 to 5 |
| Ex. 20 | 0.34 | 0.17 | 0.5 | Pure Mo | Pure Ti | | 1 to 5 |
| Ex. 21 | 0.34 | 0.10 | 0.3 | Pure Mo | Pure Ti | | 1 to 5 |
| Ex. 22 | 0.34 | 0.03 | 0.1 | Pure Mo | Pure Ti | | 1 to 5 |
| Ex. 23 | 0.34 | 0.17 | 0.5 | Pure Mo | Pure Ti | | 0 |
| Ex. 24 | 0.34 | 0.32 | 0.95 | Doped Mo | Pure Ti | | 1 to 5 |
| Ex. 25 | 0.34 | 0.02 | 0.05 | Doped Mo | Pure Ti | | 1 to 5 |
| Ex. 26 | 0.34 | 0.24 | 0.7 | 25% Re—W | Pure Ti | | 1 to 5 |
| Ex. 27 | 0.34 | 0.10 | 0.3 | 25% Re—W | Pure Ti | | 1 to 5 |
| Ex. 28 | 0.34 | 0.17 | 0.5 | 50% W—50% Mo | Pure Ti | | 1 to 5 |
| Ex. 29 | 0.34 | 0.17 | 0.5 | Doped W | Nitinol TiNi | | 1 to 8 |
| Ex. 30 | 0.34 | 0.03 | 0.1 | Doped W | Nitinol TiNi | | 1 to 8 |
| Ex. 31 | 0.34 | 0.17 | 0.5 | Doped Mo | Nitinol TiNi | | 1 to 7 |
| Ex. 32 | 0.34 | 0.17 | 0.5 | Doped W | 13% Ta—29% Nb—4.6% | β-titanium, low | 1 to 5 |
| | | | | | Zr—Ti alloy | Young's modulus | |
| Ex. 33 | 0.34 | 0.17 | 0.5 | 10% Ir—W | 13% Ta—29% Nb—4.6% | β-titanium, low | 1 to 5 |
| | | | | | Zr—Ti alloy | Young's modulus | |
| Ex. 34 | 0.34 | 0.17 | 0.5 | 10% Rh—W | 13% Ta—29% Nb—4.6% | β-titanium, low | 1 to 5 |
| | | | | | Zr—Ti alloy | Young's modulus | |
| Ex. 35 | 0.34 | 0.17 | 0.5 | 10% Ru—W | 13% Ta—29% Nb—4.6% | β-titanium, low | 1 to 5 |
| | | | | | Zr—Ti alloy | Young's modulus | |
| Ex. 36 | 0.34 | 0.17 | 0.5 | Doped W | Ti—6% Al—4% V | (α + β)-titanium, low | 1 to 5 |
| | | | | | | Young's modulus | |
| Comp. | 0.34 | 0.00 | 0 | Pure W | — | | — |
| Ex. 3 | | | | | | | |
| Comp. | 0.34 | 0.34 | 1 | Pure Ti | — | | — |
| Ex. 4 | | | | | | | |
| Comp. | 0.34 | 0.34 | 1 | Nitinol TiNi | — | | — |
| Ex. 5 | | | | | | | |
| Comp. | 0.34 | 0.32 | 0.02 | Nitinol TiNi | Copper plating | | — |
| Ex. 6 | | | | | | | |

| | | Torque transmissibility | | | | |
|---|---|---|---|---|---|---|
| | | | | Frequency of | | |
| | | Angular displacement | | discontinuous | | Pushability |
| | Yeild in wire | in output part, deg. | | rotation/ | Hysteresis | Amplitude |
| | drawing, % | Average | Variation | cycle | width, deg. | of output part, mm |
| Ex. 8 | 68 | 82 | 5 | 0 | 4 | 8.5 |
| Ex. 9 | 75 | 84 | 4 | 0 | 6 | 8.0 |
| Ex. 10 | 85 | 86 | 4 | 0 | 8 | 7.8 |
| Ex. 11 | 80 | 80 | 4 | 0 | 7 | 7.6 |
| Ex. 12 | 65 | 70 | 10 | 0 | 4 | 6.2 |
| Ex. 13 | 98 | 86 | 2 | 0 | 7 | 7.8 |
| Ex. 14 | 52 | 78 | 20 | 4 | 15 | 7.2 |
| Ex. 15 | 53 | 80 | 15 | 10 | 13 | 8.5 |
| Ex. 16 | 42 | 55 | 13 | 2 | 12 | 4.6 |
| Ex. 17 | 90 | 87 | 15 | 0 | 12 | 7.7 |
| Ex. 18 | 89 | 74 | 6 | 1 | 5 | 8.0 |
| Ex. 19 | 74 | 76 | 5 | 0 | 7 | 7.8 |
| Ex. 20 | 86 | 70 | 4 | 0 | 8 | 7.5 |
| Ex. 21 | 78 | 68 | 10 | 0 | 7 | 6.4 |
| Ex. 22 | 64 | 65 | 12 | 0 | 6 | 5.8 |
| Ex. 23 | 52 | 67 | 18 | 3 | 12 | 7.0 |
| Ex. 24 | 50 | 76 | 17 | 9 | 13 | 7.5 |
| Ex. 25 | 48 | 52 | 14 | 2 | 11 | 4.2 |
| Ex. 26 | 86 | 84 | 3 | 0 | 7 | 8.1 |
| Ex. 27 | 83 | 80 | 3 | 0 | 7 | 7.8 |
| Ex. 28 | 83 | 75 | 4 | 0 | 8 | 7.6 |
| Ex. 29 | 85 | 84 | 4 | 0 | 10 | 6.1 |
| Ex. 30 | 70 | 58 | 8 | 0 | 12 | 4.8 |
| Ex. 31 | 80 | 74 | 4 | 0 | 9 | 5.6 |
| Ex. 32 | 92 | 86 | 8 | 0 | 9 | 6.6 |
| Ex. 33 | 76 | 89 | 4 | 0 | 3 | 8.9 |
| Ex. 34 | 77 | 60 | 3 | 0 | 4 | 8.7 |
| Ex. 35 | 68 | 88 | 4 | 0 | 4 | 8.7 |
| Ex. 36 | 88 | 83 | 10 | 0 | 10 | 7.0 |
| Comp. | — | 83 | 20 | 15 | 16 | 6.8 |
| Ex. 3 | | | | | | |
| Comp. | — | 50 | 23 | 3 | 13 | 4.3 |
| Ex. 4 | | | | | | |
| Comp. | — | 83 | 4 | 0 | 15 | 3.5 |
| Ex. 5 | | | | | | |
| Comp. | — | 83 | 6 | 0 | 13 | 3.6 |
| Ex. 6 | | | | | | |

As can be seen from the table, the core wires for the guide wires of the Examples of the present invention had high torque transmissibility and pushability.

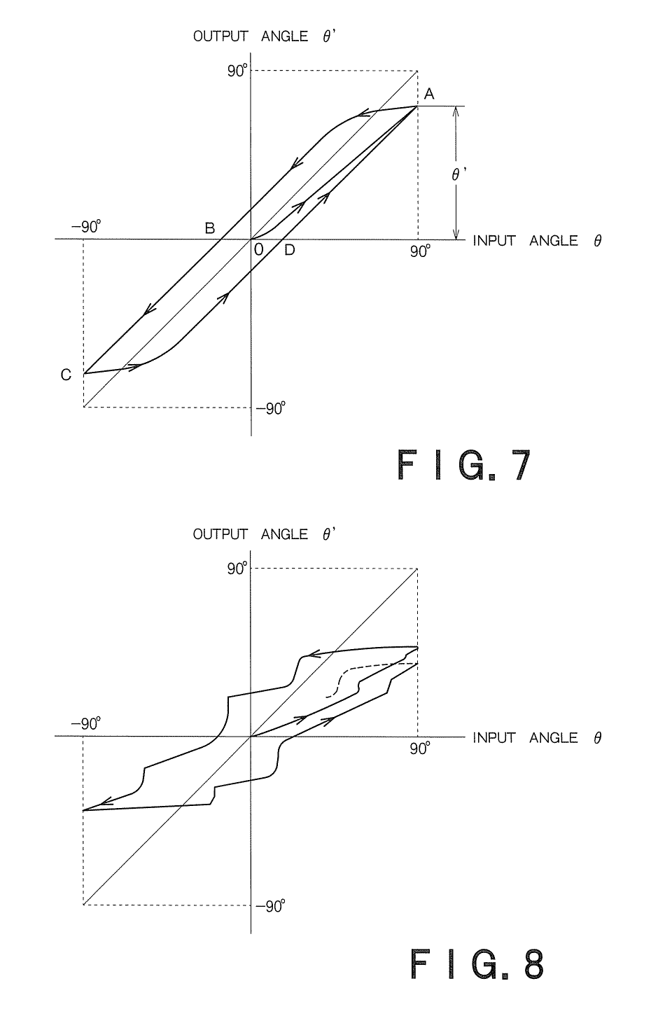

The results of the evaluation of torque transmissibility for Example 8 and the results of the evaluation of torque transmissibility for Example 24 are shown in FIGS. 7 and 8, respectively.

In FIGS. 7 and 8, the abscissa represents the angle of rotation of the input part, and the ordinate represents the angle of the rotation of the output part. In FIG. 7, the rotation of the guide wire was started from the origin (point O). The guide wire was rotated to point A where the angle of the input part became +90 degrees. Thereafter, the direction of the rotation was reversed, and the guide wire was passed through point B and was rotated to point C where the angle of the input part was −90 degree. The direction of rotation of the guide wire was again reversed, and the guide wire was passed through point D, and was rotated to point A where the angle of the input part was +90 degrees. The drawing shows (1) angle θ′ of the output angle when the angle of the input part was 90 degrees, (2) the frequency of discontinuous rotation, and (3) the width of hysteresis (angle width corresponding to the spacing between point B and point D in FIG. 7).

The frequency of discontinuous rotation is defined as follows. When rotation was applied to the input part, the occurrence of the force of constraint in the rotary motion, for example, by friction between the PTFE tube and the core wire for the guide wire sometimes causes such an unfavorable phenomenon that the rotation of the input part as such is not transmitted to the output part. The rotary motion is transmitted to the output part only when the stress developed by the rotation of the input part is higher than the force of constraint and the like. Therefore, when the torque transmissibility is poor, the output part causes discontinuous rotation, resulting in a hysteresis curve (phenomenon) as shown in FIG. 8. The index of whether the rotation is discontinuous shows that the occurrence of a level difference of not less than 3 degrees in terms of output angle is counted as one, and the number of times of occurrence of discontinuous rotation per cycle of the hysteresis cure is counted.

FIG. 8 shows an example where the number of times of discontinuous rotation is 6. For Example 24, the frequency of discontinuous rotation in Table 2 is “9” which is the average of the measured values for the 10 samples.

1. A medical guide wire comprising: a medical guide wire main body part comprising a body part and a frontal end part having a smaller wire diameter than the body part; and a coil part and a cap part provided at the frontal end part, characterized in that
the coil part comprises a wire which is a clad wire comprising a core part that is composed mainly of at least one of tungsten and molybdenum and a covering part that covers the core part and is composed mainly of titanium.

2. The medical guide wire according to claim 1, wherein a solid solution comprising at least one of tungsten and molybdenum and titanium is present at the boundary between the core part and the covering part.

3. The medical guide wire according to claim 1, wherein a solid-solution layer comprising at least one of tungsten and molybdenum and titanium is present at the boundary between the core part and the covering part.

4. The medical guide, wire according to claim 3, wherein the thickness of the solid-solution layer is not less than 0.003 time of the wire diameter in the coil part.

5. The medical guide wire according to claim 1, wherein the coil part comprises the clad wire wound by three or more turns.

6. The medical guide wire according to claim 1, wherein the core part is formed of a tungsten alloy containing at least one of rhenium, iridium, rhodium, and ruthenium.

7. The medical guide wire according to claim 1, wherein the covering part is formed of a titanium alloy comprising at least one of super elastic titanium alloys, α-titanium alloys, α+β titanium alloys, or β-titanium alloys.

8. The medical guide wire according to claim 1, wherein the young's modulus of the covering part composed mainly of titanium is not more than 140 GPa.

9. The medical guide wire according to claim 1, wherein the young's modulus of the core part composed mainly of tungsten or molybdenum is not less than 327 Gpa.

10. The medical guide wire according to claim 1, wherein the wire diameter of the coil part, D1, is not more than 0.05 mm.

11. The medical guide wire according to claim 1, wherein the wire diameter ratio between the coil part and the core part, D2/D1, is in the range of 0.1 to 0.9 wherein D1 represents the wire diameter of the coil part; and D2 represents the wire diameter of the core part.

12. The medical guide wire according to claim 1, wherein the wire diameter of the body part is not more than 0.5 mm.

13. The medical guide wire according to claim 1, wherein the length of the body part is not less than 30 cm.

14. The medical guide wire according to claim 1, wherein the body part constituting the medical guide wire main body part is a clad wire comprising a core part that is composed mainly of at least one of tungsten and molybdenum and a covering part that covers the core part and is composed mainly of titanium.
